(19) United States
(12) Patent Application Publication
(10) Pub. No.: US 2011/0081690 A1
(43) Pub. Date: Apr. 7, 2011

(54) Sustainable method for synthesizing tetrodotoxin (TTX)

(76) Inventors: Malin Strand, Stromstad (SE); Per Sundberg, Goteborg (SE)

(21) Appl. No.: 12/924,302

(22) Filed: Sep. 24, 2010

Related U.S. Application Data
(60) Provisional application No. 61/277,807, filed on Sep. 30, 2009.

(51) Int. Cl. C12P 17/18

(52) U.S. Cl. 435/119

(57) ABSTRACT

A sustainable biosynthesizing of tetrodotoxin (TTX) based on seed culture of Vibrio spp is obtained from the mucus of various species of nemerteans (ribbon worms) (phylum Nemertea). The indispensable organisms are kept alive which extends access to crude material and makes the procedure economically and ecologically sustainable.

### CROSS-REFERENCE TO RELATED APPLCIATIONS

This application claims priority from U.S. provisional application Ser. No. 61/277,807, filed Sept. 30, 2009.

### BACKGROUND OF THE INVENTION

1. Field of the Invention

The present invention relates generally to a method of extracting tetrodotoxin (TTX) from the mucus of specific species of nemertean worms. More specifically the invention relates to an ecological method of repeatedly using the worms for obtaining mucus, allowing the worms to be kept alive and sustain a continuous source for mucus production, resulting in a high yield of tetrodotoxin production.

2. Description of the Prior Art

Tetrodotoxin (TTX) is a highly potent neurotoxin and is also known as the causative agent of puffer fish poisoning. Moreover, not all species of puffer fish are toxic, and several are only weakly or moderately toxic. The toxicity of puffer fish species varies depending on the tissues or organs, geography, season of the year, and sex. TTX is not restricted to puffer fish and is widely distributed among various kinds of animals, such as the California newt Tarichi torosa, the goby Gobius criniger, Atelopus frogs, the gastropod mollusks Charonia sauliae and Babylonia japonica, the xanthid crab Atergatis floridus, the blue-ringed octopus Octopus maculosus, Astropecten starfishes, the frog shell Tutufa lissostoma, small gastropod mollusks Zeuxis siquijorensis, the Niotha clathrata) and various species of ribbon worms (Nemertea). These facts indicate that TTX-containing animals may have absorbed and accumulated TTX and its derivatives produced by several marine bacteria. The origin of TTX in marine animals has been the subject of a number of investigations. The probable mechanism of toxification of TTX-bearing animals has been discovered: Vibrio fischeri isolated from the xanthid crab Atergatis floridus and Vibrio alginolyticus isolated from the puffer fish Fugu vermicularis vermicularis produced TTX and anhydro-TTX. The number of bacterial strains reported to produce the toxin has been increasing, and most strains have been identified as members of the genus Vibrio.

Tetrodotoxin (TTX) is physiologically active by inhibiting sodium movement whereby the action potential along the nerve membranes is blocked, and has a great potential as a novel pharmaceutical drug for pain relief in terminal ill cancer patients, a local anaesthetic, and as an aid in relieving withdrawal symptoms in heroin addicts. It is estimated that 1200 g of TTX will be required by 2 million cancer patients in a one-month treatment course, whilst 400 g of TTX will be required by another 2 million drug addicts every 10 days.

Tetrodotoxin isolated from the ovaries of the puffer fish is currently the commercial source of TTX. However, the extraction yield of TTX is extremely low, about 1 g TTX per 100 Kg of ovaries, thus making it one of the highest priced natural neurotoxins. TTX can only be extracted from wild-caught fish and stocks of puffer fish have been falling due to overfishing. Furthermore, extraction based on this vulnerable source is not ecologically sustainable and will eradicate the fish stock as the demand of TTX increases unless other sources can be found. Because of the molecule's complexity it is more economical to extract tetrodotoxin directly from puffer fish ovaries, or any other biological source as here proposed.

Many researchers continue to propose new synthesis methodologies but so far these have not been found economically viable. All other hitherto known methods to extract TTX from organisms are, as mentioned before, lethal to the organism necessary for TTX production. For example as described in patent application WO 2008/102253, tissue extract from a tetrodotoxin-bearing organism is used, wherein said tissue extract is taken from organs including intestine, liver, ovary, stomach, or skin of such organism.

The method of the invention herein will overcome the abovementioned disadvantages by continuously using the same animals, resulting in a high yield of tetrodotoxin production per animal. For the long-term survival the animals are kept in an optimal milieu in aquariums with continuous inflow of deep-sea water. The mucus of the nemertean worms bears TTX-producing bacteria. For obtaining the mucus of the nemertean worms, the mucus is carefully collected by tactile stimulation. Since the animals are kept alive they sustain a continuous source for mucus production. For maintaining the capability of the bacteria to produce TTX over a long time, the mucus of the nemertean worms is continuously needed to be added to the fermentation-process.

Carroll et al (Carroll S.; McEvoy E. G.; Gibson. The production of tetrodotoxin-like substances by nemertean worms in conjunction with bacteria. Journal of Experimental Marine Biology and Ecology, Volume 288, Number 1, 25 March 2003, pp. 51-63) found evidence for V. alginolyticus-like bacteria in 10 species of free-living predatory marine nemerteans, nine of which showed bacterial growth in their epidermal mucus. The investigations were carried out on extracts made of homogenizing fresh nemertean tissues and the mucus of Lineus longissimus. Carroll speculates that to synthesize TTX, the bacteria may require some substrate either from the nemertean epidermal mucus or from within their body tissues and that perhaps Vibrio species cannot synthesize TTX without their host species. Carroll et al further suggests that the high degree of activity evidenced by the mucus of Lineus longissimus may indicate that it is in this epidermal secretion that the bulk of the substrate is found. They showed that the first but not second generation bacteria produced a sufficient amount of TTX, but they did not show or suggest that a continuously addition of nemertean mucus to the fermentation-process would solve the problem of the decreased ability of TTX-producing bacteria to produce TTX. Nor did Carroll et al. succeed to keep the worms alive by providing them a perfect milieu. Consequently they did not manage to use the worms as a continuous source for mucus production and thus a high yield of TTX extraction per worm.

The method of preparing TTX by microbial fermentation technology is already known in the art, it is for example described in WO 2008/102253 and CN1680567A. However unlike the invention herein the applications do not mention addition of mucus of nemertean worms to the fermentation process nor do they mention the long-term survival of the animals due to a continuous inflow of deep-sea water.

Thus the method of the invention herein will provide high production quantities of TTX and thus overcome the problems with using the limited resource of puffer fish, and at the same time allowing the animals to be kept alive and sustain the mucus production.

### SUMMARY OF THE INVENTION

A primary object of the present invention is provide an ecological method of extracting TTX from mucus of nemertean worms allowing the animals to be kept alive and sustain mucus production.

An object of the present invention is to provide an optimal milieu in aquariums for a long-term survival of the nemertean worms.

An object of the present invention is to provide an optimal milieu in aquariums, by supplying the aquarium with continuous inflow of deep-sea water.

An object of the present invention is to provide a method of continuously adding the mucus of nemertean worms to the fermentation-process of the TTX-production.

Other objects and advantages of the present invention will become obvious to the reader and it is intended that these objects and advantages are within the scope of the present invention.

### DETAILED DESCRIPTION OF THE INVENTION AND PREFERRED EMBODIMENTS THEREOF

The invention herein provides a sustainable method for obtaining tetrodotoxin neurotoxin or a related neurotoxin produced by a bacterial strain that is capable of producing the neurotoxin in an animal host species. The method includes the steps of growing the bacterial strain in an in vitro bacterial fermentation culture; keeping animals of the animal host species under conditions conducive for growth of the animal host species including continuous inflow of deep-sea water; collecting mucous from living animals of the animal host species; adding the collected mucous to the bacterial fermentation culture to result in neurotoxin production by the bacterial strain; isolating and purifying neurotoxin, produced by the bacterial strain, from the fermentation medium; and repeating these steps with the same animals of the animal host species, by adding freshly collected mucus from the animals of the animal host species and new fermentation medium to the bacterial fermentation culture. In the method of the invention, animals of the animal host species are kept alive as a sustained continuous source for mucus production, resulting in a high yield of neurotoxin per animal of the animal host species.

In the preferred method of the invention, Tetrodotoxin-producing bacteria such as Vibrio sp: DSM 22984 , DSM 22985, DSM 22986 and DSM 22987, in mucus from nemerteans such as Lineus longissimus and Riseriellus occultus, are added to TCBS agar plate and incubated at 25 degrees Celsius for 24-76 hours. Yellow colonies are picked up and added to a fermentation medium and allowed to grow at 25 degrees Celsius for 24-48 hours at a mechanical shaker. The tetrodotoxin is isolated and purified from the fermenter by a series of steps: washing, centrifugation, solid phase extraction in two steps, elution and finally evaporation under reduced pressure.

Alternatively revived frozen or freeze-dried tetrodotoxin-producing bacteria can be added to the fermenter medium together with mucus from nemertean worms. The frozen or freeze-dried tetrodotoxin-producing bacteria are preferably: Vibrio sp: DSM 22984 , DSM 22985, DSM 22986, DSM 22987 or any V. alginolyticus or V. gigantus.

The invention refers to tetrodotoxin and its analogues, including but limited to anhydrotetrodotoxin, tetrodaminotoxin, methoxytetrodotoxin, ethoxytetrodotoxin, deoxytetrodotoxin, and tetrodonic acid Tetrodotoxin-producing bacteria are preferably a Vibrio bacteria but most preferably Vibrio alginolyticus, Vibrio gigantus and the new and deposited Vibrio sp: DSM 22984 , DSM 22985, DSM 22986, DSM 22987. Other Vibrio species can be Vibrio nigripulchritudo, Vibrio mediterranei, Vibrio harveyi, Vibrio salmonicida, Vibrio tubiashii, Vibrio parahaemolyticus, Vibrio campbelli, Vibrio natriegens, Vibrio nereis, Vibrio carchariae, Vibrio fluvialis, Vibrio fischeri, Vibrio vulnificus, Vibrio splendidus, Vibrio orientalis, Vibrio aestuarianus, Vibrio pelagius, Vibrio wodanis, Vibrio furnissii, Vibrio proteolyticus, Vibrio ichthyoenteri, Vibrio pectenicida, Vibrio logei, Vibrio mimicus, Vibrio mytili, Vibrio rumoiensis, Vibrio anguillarum, Vibrio gazogenes, Vibrio halioticoli, Vibrio hollisae, Vibrio ordalii, Vibrio metschnkiovii or undescribed Vibrio and other species recently found associated with different nemertean species.

The mucus with tetrodotoxin-bearing organisms is obtained from any nemertean species with TTX-producing bacteria and includes species such as Lineus longissimus, Riseriellus occultus, Tubulanus annulatus, Cephalotrix spp, and preferably is from Lineus longissimus and Riseriellus occultus.

The growth medium is preferably a standard medium for the cultivation of Vibrio bacteria, preferably standard nutrient broth with sterile water replaced with oceanic water (salinity 3.1-3.4%, filter through 0.2 micrometer).

The fermenter medium comprises soy peptone, yeast extract, sugars, distilled water, or buffers. The fermenter medium is preferably a standard fermenter medium for the fermentation of Vibrio bacteria.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLE 1

### Preparation of an Optimal Milieu for a Long-Time Survival of Nemertean Worms in Aquariums.

Nemerteans are kept in glass aquaria with continuously flowing oceanic water from 45 m depth, salinity range 3.1-3.4%, temperature range 7-16° C. (ranges within a calendar year). The water flow must not fall below 2 dl/min. Water is not filtered in any way affecting quality of water. No substances are added. The aquaria are kept in darkness for 15 hrs/day. No direct light is applied except daylight for the remaining time per day. The aquaria contain shells and stones for hiding spaces for the worms.

### EXAMPLE 2

Preparation of the Mucus from a Nemertan Worm

Worms are collected from aquaria to a clean and dry glass bowl (25 ml). Tactile stimulation is carried out with a soft plastic pipette (10 ml) through rinsing the worms with 10 ml fresh sea water, taking in the same water and repeatedly rinsing the worms with the same media (sea water). Mucus production follows this stimulation procedure. Approximately 20 ml of liquid sea water containing mucus is collected with the soft plastic pipette, and transferred to a sterile vial.

### EXAMPLE 3

Biosynthesis of TTX from Mucus of Nemertean Worms

Liquid containing sea water and mucus from nemertean worms is filtered in a stepwise procedure. First the filtration is roughly through standard filter paper to exclude organic material such as algal rests or depository material. Second, the filtration is with manual pressure through a syringe filter 0.4 micrometer (any diameter filter Third, the filtration is with manual pressure through a syringe filter 0.2 micrometer (any diameter filter). All steps follow standard procedures according to the filter manufacturer.

The liquid sample then is transferred to a Solid Phase Extraction (SPE) column Evolute ABN (Manufacturer Sorbent AB, Sweden). Preparation/activation of the column (example: volume 200 mg/6 ml-50 micrometer) is done with:
1.6 ml MeOH
2. 6 ml sterile water
3. Sample application (up to a volume of 15 ml) and collect again. This procedure rinses proteins and organic molecules from the sample. In this step the proteins and other molecules stick to the column and the TTX comes out in the liquid phase. So, the liquid that leaks from the column should be collected in a vial and then proceed to application of sample on the next column.

To separate TTX and salt, the next step is a SPE column C 18 (EC) (manufacturer Sorbent AB, Sweden). Preparation/activation of column (example: volume 500 mg/6 ml-50 micrometer) is done with:
1. 2 ml MeOH
2. Apply sample (up to a volume of 15 ml)
3. Rinse with 2 ml sterile water to remove salt, with the TTX remaining on the column
4. Elute TTX with MeOH 100%, 4 ml
5. Optional: Evaporate under reduced pressure

### EXAMPLE 4

Continuous Biosynthesis of TTX from Mucus Derived of the Nemertean Worms

Mucus (see Example 2) is transferred with an inoculation loop to TCBS Vibrio selective agar (Manufacturer Fluka/Sigma) and incubated at room temperature 24-48 hours. Yellow colonies (which are Vibrio) are transferred with an inoculation loop to autoclaved standard nutrient broth made from filtered (0.2 micrometer) oceanic water (from 45 m depth) instead of sterile fresh water and then incubated at room temperature 20-24° C. for 24-56 hours continuously shaking so the medium is in constant contact with bacteria. After defined bacterial activity (as compared to control regrowth in an uninoculated vial), fresh mucus (see example 2 and 3) and new nutrient broth are added. Optionally, the mixture is transferred to a larger vial/container, and incubated shaking at room temperature for 24-48 hours. The procedure is repeated for continuous production of TTX.

Samples are centrifuged at 5000G for 30 min. The supernatant is collected and filtered through a 0.2 micrometer filter and then transferred to a SPE column (see example 3).

### EXAMPLE 5

Continuous Biosynthesis of TTX from Revived Deposited Frozen Bacteria and the Mucus of Nemertean Worms

Frozen Vibrio sp: DSM 22984 , DSM 22985, DSM 22986, and DSM 22987 are revived by warming in 4-8° C. and, when liquid, a portion is transferred to new vial containing fresh nutrient broth (according to previous examples) and incubated at room temperature (20-24° C.) for 24-56 hours, continuously shaking so the medium is in constant contact with bacteria. After defined bacterial activity (control regrowth in vial) fresh mucus (see example 2 and 3) and new nutrient broth are added. Optionally the mixture is transferred to a larger vial/container, and incubated shaking at room temperature for 24-48 hours. The procedure is repeated for continuous production of TTX.

The samples are centrifuged at 5000 G for 30 min. The supernatant is collected and filtered through a 0.2 micrometer filter and then transferred to a SPE column (see example 3).

What is claimed is:

1. A sustainable method for obtaining tetrodotoxin neurotoxin or a related neurotoxin produced by a bacterial strain that is capable of producing the neurotoxin in an animal host species, comprising
a) growing the bacterial strain in an in vitro bacterial fermentation culture;
b) keeping animals of the animal host species under conditions conducive for growth of the animal host species including continuous inflow of deep-sea water;
c) collecting mucous from living animals of the animal host species grown in step b);
d) adding the collected mucous to the bacterial fermentation culture to result in neurotoxin production by the bacterial strain;
e) isolating and purifying neurotoxin, produced by the bacterial strain, from the fermentation medium;
f) repeating step b) through step e) with the same animals of the animal host species, by adding freshly collected mucus from the animals of the animal host species and new fermentation medium to the bacterial fermentation culture;
wherein animals of the animal host species are kept alive as a sustained continuous source for mucus production, resulting in a high yield of neurotoxin per animal of the animal host species.

2. The method of claim 1, wherein the bacterial strain is a strain of the genus Vibrio.

3. The method of claim 1, wherein the bacterial strain is selected from the group consisting of Vibrio fischeri, Vibrio alginolyticus, Vibrio gigantus, Vibrio sp: DSM 22984 , DSM 22985, DSM 22986, and DSM 22987, Vibrio nigripulchritudo, Vibrio mediterranei, Vibrio harveyi, Vibrio salmonicida, Vibrio tubiashii, Vibrio parahaemolyticus, Vibrio campbelli, Vibrio natriegens, Vibrio nereis, Vibrio carchariae, Vibrio fluvialis, Vibrio fischeri, Vibrio vulnificus, Vibrio splendidus, Vibrio orientalis, Vibrio aestuarianus, Vibrio pelagius, Vibrio wodanis, Vibrio furnissii, Vibrio proteolyticus, Vibrio ichtliyoenteri, Vibrio pectenicida, Vibrio logei, Vibrio mimicus, Vibrio mytili, Vibrio rumoiensis, Vibrio anguillarum, Vibrio gazogenes, Vibrio halioticoli, Vibrio hollisae, Vibrio ordalii, Vibrio metschnkiovii or undescribed Vibrio and other neurotoxin-producing bacterial species in nemertean species.

4. The method of claim 1, wherein the bacterial strain is selected from the group consisting of Vibrio fischeri, Vibrio alginolyticus, Vibrio gigantus, and Vibrio sp: DSM 22984 , DSM 22985, DSM 22986, and DSM 22987.

5. The method of claim 1, wherein the mucus is collected after tactile stimulation of the host species.

6. The method of claim 1, wherein the animal host species is a species of nemertean worms.

7. The method of claim 1, wherein the bacteria are isolated from mucus from nemerteans such as Lineus longissimus and Riseriellus occultu.

8. The method of claim 1, wherein the neurotoxin is purified by steps of washing, centrifugation, solid phase extraction in two steps, elution and evaporation under reduced pressure.

9. The method of claim 1, wherein the fermentation medium is a standard medium for growing Vibrio.

10. The method of claim 1, wherein the bacterial strain comprises revived freeze-dried tetrodotoxin-producing bacteria.

11. The method of claim 1, wherein the neurotoxin is selected from the group consisting of tetrodotoxin, anhydrotetrodotoxin, tetrodaminotoxin, methoxytetrodotoxin, ethoxytetrodotoxin, deoxytetrodotoxin, and tetrodonic acid.

12. The method of claim 1, wherein the neurotoxin is tetrodotoxin.

13. The method of claim 1, wherein the nemertean species is selected from the group consisting of Lineus longissimus, Riseriellus occultus, Tubulanus annulatus, and Cephalotrix spp.

14. The method of claim 1, wherein the nemertean species is selected from the group consisting of Lineus longissimus and Riseriellus occultus.
